(19) United States
(12) Patent Application Publication
(10) Pub. No.: US 2026/0239514 A1
(43) Pub. Date: Aug. 13, 2026

(54) AN ILLUMINATION SYSTEM

(71) Applicant: SIGNIFY HOLDING B.V., Eindhoven (NL)

(72) Inventors: CURTIS ALLEN LEYK, ALBANY, MN (US); JACK TIEBERG, PLYMOUTH, MN (US); AARON BENJAMIN STEPHAN, CHANHASSEN, MN (US)

(21) Appl. No.: 19/155,985

(22) PCT Filed: Feb. 5, 2024
(86) PCT No.: PCT/EP2024/052790

§ 371 Date: Aug. 13, 2025

Related U.S. Application Data
(60) Provisional application No. 63/445,454, filed on Feb. 14, 2023.

(30) Foreign Application Priority Data
Mar. 28, 2023 (EP) 23164734.8

(51) Int. Cl. H05B 47/155; A01K 31/18; A01K 41/02; A61M 21/02; H05B 47/12; H05B 47/125; H05B 47/13; H05B 47/16; H05B 47/165

(52) U.S. Cl. CPC H05B 47/155; A01K 31/18; A01K 41/02; A61M 21/02; H05B 47/12; H05B 47/125; H05B 47/13; H05B 47/16; H05B 47/165; A61M 2250/00

(57) ABSTRACT

The invention provides an illumination system comprising: a visible light source configured to emit visible light; a blue light source configured to emit monochromatic blue light; a controller configured to control the visible light source according to a predetermined circadian schedule for entraining a circadian rhythm of a bird; wherein the predetermined circadian schedule consists of a predetermined period of light and a predetermined period of dark, wherein the controller is configured to: (i) control the visible light source to emit the visible light for the predetermined period of light, and not to emit the visible light for the predetermined period of dark; (ii) control the blue light source to emit the monochromatic blue light at a blue light intensity for at least one subperiod within the predetermined period of dark.

### FIELD OF THE INVENTION

The invention relates to an illumination system. The invention further relates to a method of illuminating a bird. The invention further relates to an incubator.

### BACKGROUND OF THE INVENTION

Economics of scale and production efficiency have driven poultry farming forward. Nowadays, poultry farming has become more and more concerned with improving animal health and wellbeing as well. This is a clear challenge.

The rearing of poultry (or: birds) starts at fertilization. The fertilized eggs are typically incubated in an incubator, wherein the bird embryo develops into a grown chick within the egg. The egg hatches usually after 21 days for chicken.

Post hatch, the hatchling/chick is then grown out further to a full-grown bird, by for example regulating its daily sleep-awake cycle (i.e. the circadian rhythm) with artificial lighting. Such lighting is generally characterized by providing a 24-hour cycle consisting of a period of light (or: day), when the artificial lighting is on, and a period of dark (or: night), when the artificial lighting is off. This entrains a circadian rhythm of the bird.

Extending the period of light (or: day) is thereby considered beneficial for increasing the pace of broiler growth, but detrimental for animal health and welfare, as the poultry also need a certain amount of sleep to prevent sleep deprivation and/or to physically recover. Hence, many jurisdictions around the world regulate artificial lighting in poultry farming, especially a minimum number of hours for said period of dark, to safeguard animal health and wellbeing.

For example: The Canadian ‘Code of Practice for the Care and Handling of Hatching Eggs, Breeders, Chickens and Turkeys’ recommends a dark period of minimum 4 hours by day 5 of bird placement, and from day five of placement through to no sooner than seven days prior to catching, birds kept in barns must have a dark period of at least four straight hours in each 24-hour period.

For example: Council Directive 2007/43/EC of the European Union defines that within seven days from the time when the chickens are placed in the building and until three days before the foreseen time of slaughter, the lighting must follow a 24-hour rhythm and include periods of darkness lasting at least six hours in total, with at least one uninterrupted period of darkness of at least four hours, excluding dimming periods.

Hence, to safeguard the health and wellbeing of poultry, and to retain a healthy melatonin cycle, it is generally recognized that it is beneficial to provide birds with a minimum number of hours of dark.

However, during the period of dark, awake birds do not eat and drink, due to the inability to find food in the dark. Therefore, their gastrointestinal tracts are nearly empty by ‘morning’when the artificial lighting is turned back on. This may be disadvantageous. Namely, upon the start of the period of light (or: day), the birds are very hungry and exhibit a voraciously eating behaviour. It is found that this causes a sudden influx of large amounts of feed into the digestive tract, which may then lead to inconsistent feeding, gastrointestinal upset, or even disease in susceptible birds.

All in all, in poultry farming and/or the rearing of birds in particular, there is a clear need to improve the health and wellbeing of said birds, while maintaining growth targets, and without disturbing their circadian rhythm.

### SUMMARY OF THE INVENTION

The invention is set out in the appended independent claims and dependent claims.

It is an object of the invention to provide an improved illumination system, which at least alleviates the problems and disadvantages mentioned above. Thereto, the invention provides an illumination system comprising: a visible light source configured to emit visible light; a blue light source configured to emit monochromatic blue light; a controller configured to control the visible light source according to a predetermined circadian schedule for entraining a circadian rhythm of a bird; wherein the predetermined circadian schedule consists of a predetermined period of light and a predetermined period of dark, wherein the controller is configured to: (i) control the visible light source to emit the visible light for the predetermined period of light, and not to emit the visible light for the predetermined period of dark; (ii) control the blue light source to emit the monochromatic blue light at a blue light intensity for at least one subperiod within the predetermined period of dark.

Proprietary research of ONCE/Signify has determined that dim monochromatic blue light does not disrupt the melatonin cycle (i.e. melatonin production) or the circadian rhythm activity of birds. Said birds are chicken in particular. The present invention leverages this insight, because the monochromatic blue light is emitted at the blue light intensity for at least one subperiod within the predetermined period of dark. This enables birds to feed and drink more effectively within the dark/night period of their circadian rhythm, but without disrupting their entrained circadian rhythm (i.e. day/night cycle). The results show an increased weight gain, improved Feed Conversion Ratio, and reduced mortality for post-hatch birds.

More specifically: The illumination system according to the invention comprises a visible light source, a blue light source, and a controller. The illumination system may be a luminaire. The controller controls the visible light source to emit the visible light for a predetermined period of light, and not to emit the visible light for a predetermined period of dark, thereby entraining a circadian rhythm of the bird. Such a predetermined circadian schedule is known to be advantageous for rearing birds, and the productivity thereof.

However, birds are generally known to be hungry when awake. This may be particularly relevant for broiler chicken. Therefore, when entrained a circadian rhythm, birds that are already awake during the predetermined period of dark, when the visible light source is not emitting any visible light, may clearly experience hunger, but may not be able to feed due to the dark. This is a clear disadvantage during the predetermined period of dark itself, but-as mentioned above-also renders gastrointestinal and behavioural problems when the predetermined period of light commences.

Hence, the controller according to the invention controls the blue light source to emit monochromatic blue light at the blue light intensity for at least one subperiod within the predetermined period of dark. The monochromatic blue light may be characterized as comprising at least one dominant peak (or: local maximum) within the wavelength range of 405-480 nm.

The present invention is therefore advantageous, because the emitted monochromatic blue light enables (awaken) birds to still see (the environment) during the predetermined period of dark, and thereby enable to more efficiently feed and drink during the dark, without the entrained circadian rhythm (i.e. day/night cycle) and their melatonin cycle are being disturbed.

Consequently, the birds still maintain their normal circadian rhythm, and thereby grow as scheduled, but with less stress of starvation during the predetermined period of dark. Moreover, the food intake of the (flock of) birds may be more evenly spread throughout the entire 24-hour cycle of the predetermined circadian schedule. Even further, when the predetermined period of light commences after the predetermined period of dark, the (flock of) birds may be less voraciously feeding, rendering less (aggressive) crowding, bruises, and injuries.

The present invention is also advantageous pre-hatch, during the incubation phase. Namely, it has been found that incubating with the predetermined circadian schedule according to the invention, wherein monochromatic blue light is emitted at the blue light intensity for at least one subperiod within the predetermined period of dark, has rendered an improved hatchability of fertile eggs and increased weight of hatched chicks.

All in all, the illumination system according to the present invention improves the health and wellbeing of birds, without disrupting an entrained circadian rhythm. Hence, in other words, there is clear merit in providing dim monochromatic blue light during at least part of a scotopic phase of a predetermined circadian schedule.

According to the invention, the controller controls the blue light source to emit the monochromatic blue light at a blue light intensity for at least one subperiod within the predetermined period of dark. Said monochromatic blue light is thereby dim monochromatic blue light. Hence, in an embodiment, the blue light intensity may at most be 5 lux. The blue light intensity is most preferably at most 1 lux. For example, the blue light intensity is at most 0.5 lux. For example, the blue light intensity may be between 0.01 and 0.25 lux. Such embodiments may be advantageous, because a blue light intensity above 5 lux may awake a sleeping bird, while dim levels of monochromatic blue light below 5 lux, in particular below 1 lux, may be suitable for enabling visibility of the bird, while not disturbing the entrained circadian rhythm (i.e. day/night cycle) and melatonin cycle, according to the invention.

In an embodiment, the predetermined period of light is twelve hours, and wherein the predetermined period of dark is twelve hours.

Other examples may be envisioned similarly, such as the predetermined period of light being between six and eighteen hours, wherein the predetermined period of dark being between six and eighteen hours, wherein the predetermined period of light and predetermined period of dark together constitute a full-day period of 24-hours.

In an embodiment, the visible light comprises a dominant peak at a wavelength range between 450-480 nm and a dominant peak at a wavelength range between 515-545 nm.

In an embodiment, the controller is configured to: (i) control the visible light source to emit the visible light at a visible light intensity for the predetermined period of light, wherein the visible light intensity is at least a factor 20 higher than the blue light intensity, such as for example a factor 50.

In an embodiment, the at least one subperiod is one subperiod, wherein said one subperiod lasts the whole predetermined period of dark. Such an embodiment is advantageous, as birds are still able to see during the whole predetermined period of dark and are thereby enabled to more efficiently feed and drink during the whole predetermined period of dark, without the entrained circadian rhythm (i.e. day/night cycle) and their melatonin cycle are being disturbed.

In a different embodiment, a subperiod of the at least one subperiod ends at a same time as the predetermined period of dark and lasts for a duration equal to at most a quarter of the predetermined period of dark. Such an embodiment is advantageous, because the monochromatic blue light is emitted in a subperiod that precedes, and substantially abuts with, the beginning of the predetermined period of light. Consequently, since birds will have had the opportunity to feed during the predetermined period of dark due to the emitted monochromatic blue light, that is visible to the birds, the above-mentioned problems are mitigated at the beginning of the predetermined period of light, when the visible light is emitted again. This improves the health and wellbeing of the birds.

The invention is characterized in that, the illumination system is arranged for illuminating a flock of birds; wherein the illumination system comprises a sensor unit configured to detect, during the predetermined period of dark, a property indicative of at least one bird of the flock of birds being awake; wherein the controller is configured to: control the blue light source to emit the monochromatic blue light at the blue light intensity for a subperiod within the predetermined period of dark when said property is detected.

Alternatively, the invention is characterized in that, the illumination system is arranged for illuminating a flock of birds; wherein the illumination system comprises a sensor unit configured to detect, during the predetermined period of dark, a property indicative of at least one bird of the flock of birds being awake; wherein the controller is configured to: determine a number of birds being awake based on said property, and control the blue light source to emit the monochromatic blue light at the blue light intensity for a subperiod within the predetermined period of dark when said number of birds being awake exceeds a predefined threshold number. Such an embodiment may be advantageous, because the monochromatic blue light is only provided at the blue light intensity when a threshold number of birds are being awake, thereby preventing the monochromatic blue light to be unnecessarily consuming energy for only a limited number of birds. In examples, said predefined threshold number may be at least ten. Alternatively, said predefined threshold number may be at least a hundred, or at least two hundred.

Hence, when said number of birds being awake exceeds a predefined threshold number, the blue light source is controlled to emit the monochromatic blue light at the blue light intensity for a subperiod within the predetermined period of dark. This subperiod may be considered the hold time for which the monochromatic blue light is kept on after detecting said threshold number of birds being awake.

Said subperiod may last for a duration equal to at most a half of the predetermined period of dark.

For example, if the predetermined period of dark is 2 hours, the subperiod may last for at most 1 hour. Other values may be envisioned similarly. Alternatively, in aspects, said subperiod may last for a duration equal to at most a quarter of the predetermined period of dark, or at most a tenth of the predetermined period of dark, or between a tenth and a fifth of the predetermined period of dark. Said subperiod may comprise a duration tailored for example to the desired feed intake of a bird of the flock of birds during the predetermined period of dark.

In embodiments, the sensor unit may comprise at least one of: a camera, a thermal camera, a microphone, a motion sensor, a sensor arrangement for radiofrequency-based sensing, a PIR sensor, a thermopile array, a Single Pixel Thermopile, a range sensor, a VOC sensor, a pressure sensor.

For example, the camera may detect an image of at least one sleeping bird directly. For example, the motion detector may detect motion of at least one bird, which motion may be indicative of the at least one bird being awake. For example, the microphone detector may detect sound level or noise of at least one bird, which sound level or noise may be indicative of the at least one bird being awake. Alternatively, other properties of sound may be detected, such as audio footprints of awake birds. For example, the VOC detector may detect dust levels or concentrations caused by the at least one bird, which dust level may be indicative of the at least one bird being awake. For example, the thermopile array may detect a heat signature of at least one bird being active, which heat signature may be indicative of the at least one bird being awake.

In an embodiment, the controller is configured to determine the predetermined circadian schedule. In an embodiment, the controller is configured to obtain an input signal indicative of the predetermined circadian schedule, and to determine the predetermined circadian schedule based on said input signal. Said input signal may e.g. be a user input signal. The controller may receive or retrieve said input signal. The controller may be communicably coupled to a user interface device, wherein the user interface device may be configured to receive a user input indicative of said predetermined circadian schedule and generate said input signal based on the user input. The user interface may be comprised by the illumination system according to the invention.

It is a further object of the invention to provide a method, which at least alleviates the problems and disadvantages mentioned above. Thereto, the present invention provides a method of illuminating a bird during at least one growth phase, wherein the method comprises: emitting visible light for a predetermined period of light during a full-day period, and not to emit visible light for a predetermined period of dark during the remainder of said full-day period, so as to entrain a circadian rhythm of the bird; emitting monochromatic blue light at a blue light intensity for at least one subperiod within the predetermined period of dark. The method further comprises: detecting, during the predetermined period of dark, a property indicative of at least one bird of the flock of birds being awake; controlling the blue light source to emit the monochromatic blue light at the blue light intensity for a subperiod within the predetermined period of dark when said property is detected; OR determining a number of birds being awake based on said property, and controlling the blue light source to emit the monochromatic blue light at the blue light intensity for a subperiod within the predetermined period of dark when said number of birds being awake exceeds a predefined threshold number. The advantages and/or embodiments applying to the illumination system according to the invention may also apply mutatis mutandis to the method according to the invention.

In an embodiment, blue light intensity is at most 5 lux. In an embodiment, the at least one subperiod is one subperiod, wherein said one subperiod lasts the whole predetermined period of dark.

In an embodiment, the predetermined period of light is twelve hours, and wherein the predetermined period of dark is twelve hours.

Other examples may be envisioned similarly, such as the predetermined period of light being between six and eighteen hours, wherein the predetermined period of dark being between six and eighteen hours, wherein the predetermined period of light and predetermined period of dark together constitute a full-day period of 24-hours.

In an embodiment, the visible light comprises a dominant peak at a wavelength range between 450-480 nm and a dominant peak at a wavelength range between 515-545 nm. Such a visible light represents the trademarked Junglite light recipe of Signify/ONCE.

In an embodiment, the at least one growth phase is at least one of: a pre-hatch phase, a hatching phase, a grow-out phase, a post-hatch phase.

The steps of the method according to the invention may be performed for each day of said at least one growth phase. Hence, in an embodiment, each growth phase of the at least one growth phase may comprise a range of days, wherein the method is repeatedly performed for each day in said range of days.

Thereby, said incubation stage may be defined by a first range of days, the first range of days being between zero and thirty days from fertilization of the bird (i.e. the bird embryo) or from placing an egg containing the bird in an incubator. For example, said first range of days may be between zero and twenty-one days from fertilization of the bird (i.e. the bird embryo) or from placing an egg containing the bird in an incubator, when e.g. dealing with chicken. For example, said first range of days may be between zero and twenty-eight days from fertilization of the bird (i.e. the bird embryo) or from placing an egg containing the bird in an incubator, when e.g. incubating turkey eggs.

Said hatching phase may be defined by a second range of days, the second range of days being between eighteen and twenty-five days from fertilization of the bird (i.e. the bird embryo) or from placing an egg containing the bird in an incubator.

Said grow-out phase may be defined by a third range of days, the third range of days being between zero and fourteen days from hatching of said bird.

Said post-hatch phase may be defined by a fourth range of days, the fourth range of days being between zero and fifty days from hatching of said bird, preferably between fourteen and fifty days from hatching said bird.

For example, as a light recipe, the method of illuminating the bird during at least one growth phase may comprise: (I) during an incubation stage, the incubation stage being between zero and twenty-one days from fertilization of the bird or from placing an egg containing the bird in an incubator, performing the steps of (i) emitting visible light for a predetermined period of light during a full-day period, and not to emit visible light for a predetermined period of dark during the remainder of said full-day period, so as to entrain a circadian rhythm of the bird; (ii) emitting monochromatic blue light at a blue light intensity for at least one subperiod within the predetermined period of dark; AND/OR (II) during a post-hatch phase, the post-hatch stage being between fourteen and fifty days from hatching of the bird, performing the steps of (i) emitting visible light for a predetermined period of light during a full-day period, and not to emit visible light for a predetermined period of dark during the remainder of said full-day period, so as to entrain a circadian rhythm of the bird; (ii) emitting monochromatic blue light at a blue light intensity for at least one subperiod within the predetermined period of dark; wherein the blue light intensity is at most 5 lux; wherein the at least one subperiod is one subperiod, wherein said one subperiod lasts the whole predetermined period of dark; wherein the predetermined period of light is twelve hours, and wherein the predetermined period of dark is twelve hours.

Throughout the application, said bird may be at least one bird, such as a plurality of birds. A plurality of birds may e.g. be a flock of birds, such as a flock of chicken. Thereby, the term bird also includes the bird embryo. The bird embryo being defined as: a bird in its earliest stages of development following cleavage of the zygote and ending at hatching.

The bird is preferably a chicken. However, said birds or flock of birds may alternatively be a flock of one or more of: Turkeys, Ducks, Geese, Pheasant, Quail, Guinea Fowl, Heritage Breed Chickens, Pet Birds, Songbirds.

In aspects, said visible light source may comprise a plurality of monochromatic light sources, wherein a monochromatic light source of said plurality of monochromatic light sources is the blue light source. Hence, the visible light source may comprise the blue light source. For example, the visible light source may be a LED light engine comprising a Red LED, a Green LED, and a Blue LED, all together rendering the visible light, while the Blue LED controlled in isolation renders the monochromatic blue light.

Said visible light source may be phrased as a visible lighting system, throughout. Said blue light source may be phrased as a blue lighting system, throughout. The visible light source and the blue light source may be embodied within a same housing of a same lighting apparatus, for example a luminaire.

Said predetermined period of light may be phrased as period of light. Said predetermined period of light may be phrased as predetermined period of day. Said predetermined period of dark may be phrased as period of dark. Said predetermined period of dark may be phrased as predetermined period of night.

The predetermined period of light may comprise a time duration of light. The predetermined period of dark may comprise a time duration of dark. The time duration of light and/or the time duration of dark may be set to values corresponding to the desired rearing the (flock of) birds. For example: Said duration of light may for example be 20 hours. Said duration of dark may then for example be 4 hours. The predetermined period of light and the predetermined period of dark may be recurring (on a 24 hour circadian cycle). For example, as partly mentioned before: Said duration of light may for example be 12 hours. Said duration of dark may then for example be 12 hours. The predetermined period of light and the predetermined period of dark may be recurring (on a 24 hour circadian cycle).

In aspects, said visible light may be within the visible wavelength range of poultry. In aspects, said visible light may be within the visible wavelength range between 380-750 nm. In aspects, said visible light may be white light.

In aspects, said visible light may comprise a dominant peak within the wavelength range of red light, a dominant peak within the wavelength range of green light, and a dominant peak within the wavelength range of blue light. Said dominant peak may be defined as a local maximum within the visible light spectrum.

In aspects, said visible light may comprise a visible light characteristic. Said visible light characteristic may e.g. be a light intensity. Thereby, in aspects, throughout the application, said phrasing of ‘not emitting said visible light’ may include not emitting said visible light comprising said visible light characteristic. Hence, during the predetermined period of dark, visible light may be emitted not comprising said visible light characteristic. For example, said visible light (e.g. white light) may comprise a maximum visible light intensity in the predetermined period of light, while in the predetermined period of dark the visible light is either off altogether, or is dimmed to only 1% of said maximum visible light intensity. The visible light source may for example be configured to emit the visible light at a visible light intensity, wherein the visible light intensity is at least 5 lux, preferably at least 10 lux, most preferably at least 20 lux.

In aspects, the illumination system according to the invention is configured to illuminate a space for rearing the bird, wherein the visible light source is configured to emit said visible light into the space, wherein the blue light source is configured to emit the monochromatic blue light into the space.

In aspects, the space is an indoor space, the system comprises a first feed dispensing means arranged at a first region of the indoor space; wherein the controller is configured to control the blue light source to emit the monochromatic blue light at the blue light intensity for a subperiod within the predetermined period of dark to illuminate the first region of the indoor space.

Yet in a further aspect, wherein the bird is a flock of birds, wherein the space is an indoor space, the system comprises a first feed dispensing means arranged at a first region of the indoor space; wherein the controller is configured to control the blue light source to emit the monochromatic blue light at the blue light intensity for a subperiod within the predetermined period of dark to illuminate the first region of the indoor space. Hence, the first region in which first feed dispensing means may be illuminated with the monochromatic blue light, such that particularly the feed dispensing means becomes visible for birds for the subperiod within the predetermined period of dark.

In aspects, the space may be an indoor space, the controller may be configured to control the blue light source to emit the monochromatic blue light at a first blue light intensity during a first subperiod within the predetermined period of dark to illuminate a first region of the indoor space; wherein the controller is configured to control the blue light source to emit the monochromatic blue light at a second blue light intensity during a second subperiod within the predetermined period of dark to illuminate a second region of the indoor space; wherein the first subperiod and the second subperiod are non-overlapping in time; wherein the first region of the indoor space is different from the second region of the indoor space. Such an embodiment is advantageous, as it allows to provide the monochromatic blue light at different subperiods to different regions of the indoor space. This may also enable that parts of the indoor space may consecutively be provided with the monochromatic blue light according to the present invention, so as to control the feeding and drinking behaviour of the flock of birds more precisely for the predetermined period of dark.

In further aspects thereof, the system comprises a first feed dispensing means and a second feed dispensing means; wherein first feed dispensing means is arranged in the first region, wherein the second feed dispending means is arranged in the second region. Such an embodiment is advantageous, as different feed dispensing means may be illuminated during different subperiods within the predetermined period of dark, so as to visualize said different feed dispensing means for the birds of the flock of birds during the predetermined period of dark.

In aspects, the invention provides an illumination system arranged to illuminate an indoor space for rearing birds, wherein the illumination system comprises: a visible light source configured to emit visible light; a blue light source configured to emit monochromatic blue light; a controller configured to control the visible light source according to a predetermined circadian schedule, wherein the predetermined circadian schedule contains a predetermined period of light and a predetermined period of dark, so as to entrain a circadian rhythm of the birds; wherein the controller is configured to: (i) control the visible light source to emit the visible light for the predetermined period of light, and not to emit the visible light for the predetermined period of dark; (ii) control the blue light source to emit the monochromatic blue light at a blue light intensity for at least one subperiod within the predetermined period of dark. The illumination system may be a luminaire.

In aspects, the invention provides a method of illuminating at least one bird (or egg), wherein the method comprises illuminating at least one bird (or egg) with monochromatic blue light during at least part of a scotopic phase of a predetermined circadian schedule. The monochromatic blue light may have an intensity of at most 1 lux.

### BRIEF DESCRIPTION OF THE DRAWINGS

The invention will now be further elucidated by means of the schematic non-limiting drawings:

FIG. 1 depicts schematically an embodiment of an illumination system according to the invention;

FIG. 2 depicts schematically an embodiment of an operation of the illumination system depicted in FIG. 1;

FIG. 3 depicts schematically an embodiment of an operation of the illumination system depicted in FIG. 1;

FIG. 4 depicts schematically an embodiment of an illumination system according to the invention;

FIG. 5 depicts schematically an embodiment of an illumination system according to the invention;

FIG. 6 depicts schematically a method according to the invention;

FIG. 7 depicts schematically a method according to the invention;

FIG. 8 depicts schematically an incubator according to the invention.

### DETAILED DESCRIPTION OF THE EMBODIMENTS

There is a clear need in poultry farming to improve the health and wellbeing of birds, while maintaining growth targets, and without disturbing the entrained circadian rhythm of the birds. The present invention resolves the disadvantages and problems mentioned in the background section.

The present invention is stooled on proprietary research of ONCE/Signify, that has determined that providing dim monochromatic blue light in the ‘night’ period (or: scotopic period) of a circadian schedule does not disrupt the melatonin cycle or circadian rhythm of birds. This proprietary light recipe results in a heavier bird and improved Feed Conversion Rate (FCR), while not affecting the entrained circadian rhythm of the birds.

The results also indicate that incubating fertile eggs with said light recipe, wherein monochromatic blue light is emitted at the blue light intensity for at least one subperiod within the predetermined period of dark, renders an improved hatchability (Hatch of Fertile, HOF) of the eggs and increased weight of hatched chicks.

More specifically: The research used a circadian schedule of 12 hours of light and 12 hours of night. Eggs are thereby incubated with a dim monochromatic blue light in the night period. The research found for example that incubating broiler (Ross 308) eggs in an incubator utilizing such a daily schedule comprising 12-hour visible light (i.e. light/day) and 12-hour monochromatic blue light (i.e. dark/night) results in an increased Hatch of Fertile (HOF) and heavier chicks at hatch, versus a 24-hour light incubation as control. Namely, this results in a 2.3% increase in hatched chick rate, a 2.13% increase in hatched chick weight (47.7 g vs. 46.5 g), and a significant increase in the Hatch of Fertile (HOF) (90.8% vs. 78.1%).

This incubation recipe, in combination with using dim monochromatic blue light in the night period of the grow out phase, beginning at day 14 of grow out (i.e. day 14 after hatch), resulted in a 2.25% increase in weight and significant improvement in Feed Conversion Rate in 2 of 2 trials done (1.6355 vs 1.6898 at day 43 in trial 1, p=0.04; and 1.7085 vs 1.7809 at day 49 in trial 2, p=0.025).

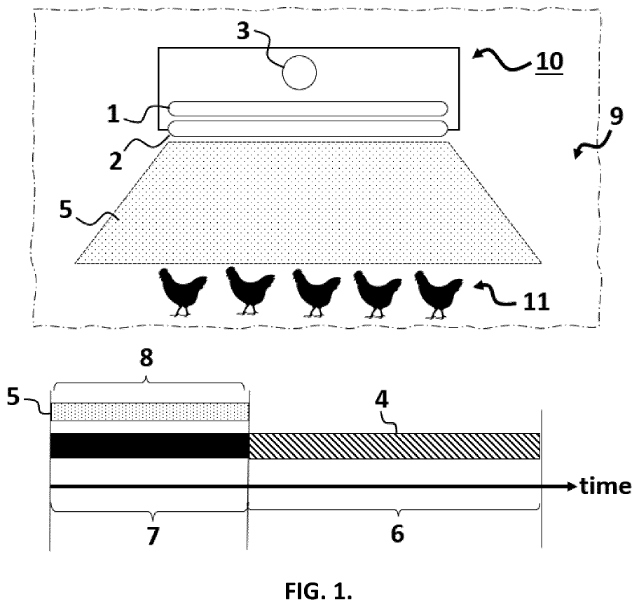

FIG. 1 depicts schematically, by non-limiting example, an illumination system 10 according to the invention. The illumination system 10 comprises a visible light source 1, a blue light source 2, and a controller 3.

The controller 3 controls, in operation, the visible light source 1 and the blue light source 2. Here, the blue light source 2 and the visible light source 1 are depicted to be part of a same luminaire, or same luminaire housing, within which also the controller is accommodated. Alternatively, said visible light source and said blue light source may be separate lighting devices, which may be controlled by a remotely arranged controller.

The visible light source 1 is configured to emit visible light. More specifically, the controller 3 controls the visible light source 1 according to a predetermined circadian schedule for entraining a circadian rhythm of a bird. The illumination system 10 according to the invention is thus configured to entrain a circadian rhythm of a bird by controlling said visible light source 1 according to the predetermined circadian schedule. Said bird may also be a bird (embryo) within an egg.

The predetermined circadian schedule (recurringly) consists of a predetermined period of light 6 and a predetermined period of dark 7. This entrains the circadian rhythm of said bird. Here, the controller 3 controls to emit the visible light 4 for the predetermined period of light 6, and not to emit the visible light for the predetermined period of dark 7.

Here, merely as an example, the predetermined period of dark comprises a duration of 4 hours, while the predetermined period of light comprises a duration of 20 hours, (in total). Other alternative schedules of light and dark may be envisioned similarly, such as for example a predetermined period of light of 12 hours and a predetermined period of dark of 12 hours.

Here, the illumination system 10 is arranged in a space 9 for rearing a bird. The space 9 is an indoor space. The space comprises a flock of birds 11, such as broiler chicken, but may alternatively be any other bird suitable for rearing.

The illumination system 10 illuminates, in operation, the indoor space 9 and thereby said flock of chicken 11. Hence, the visible light source 1 is able to render artificial lighting in the agriculture space 9 for entraining a circadian rhythm of said flock of birds 11, wherein said circadian rhythm is characterized by a 24-hour cycle comprising alternating periods of light (or: day) and periods of dark (or: night).

However, during such periods of dark, awaken birds in the flock of birds 11 may not be able to eat and drink, due to their inability to find food in the dark. This results in that their gastrointestinal tracts are nearly empty when the period of light (or: day) commences (again). Even after the period of light 6 commences (again), because the flock of birds 11 may be very hungry, the flock of birds 11 may exhibit a voraciously eating behaviour and may cause harmful crowding (e.g. at feeding throughs and water nipples).

Therefore, still referring to FIG. 1, the illumination system 10 according to the invention comprises the blue light source 2. The blue light source 2 is configured to emit monochromatic blue light 5 at a blue light intensity. Here, the monochromatic blue light 5 comprises a peak wavelength within the wavelength range of 405-480 nm. In other words, the monochromatic blue light comprises at least one dominant peak within the wavelength range of 405-480 nm.

More specifically, the controller 3 controls the blue light source 2 to emit the monochromatic blue light 5 at a blue light intensity for at least one subperiod 8 within the predetermined period of dark 7.

Here, the at least one subperiod 8 is one subperiod 8. This one subperiod 8 lasts substantially the whole predetermined period of dark 7. Namely, in the present example, said one subperiod 8 also comprises a duration of 4 hours. Consequently, during the predetermined period of dark 7 the flock of birds 11 are provided with said monochromatic blue light 5 at the blue light intensity.

Consequently, the illumination system 10 effectuates an increased health and wellbeing of the flock of birds 11. Namely: Proprietary research of ONCE/Signify has determined that monochromatic blue light does not disrupt the circadian activity (i.e. daily rhythms) or melatonin production of birds, i.e. chickens in particular.

Hence, since the blue light source 2 is controlled to emit the monochromatic blue light 5, birds of the flock of birds 11—when awaken and hungry-are still able to see within the space 9 and are thereby able to more efficiently feed and drink during the predetermined period of dark 7, without the entrained circadian rhythm (i.e. day/night cycle) and their melatonin cycle are being disturbed.

Consequently, the birds still maintain their normal circadian rhythm, and thereby grow as scheduled, but with less stress of starvation during the predetermined period of dark 7, and with less harmful behaviour when the predetermined period of light 6 commences after the predetermined period of dark 7.

Moreover, still referring to FIG. 1, the blue light intensity of the monochromatic blue light is at most 5 lux. In the present embodiment, as an example, the blue light intensity is 0.5 lux. Said blue light intensity is selected such, in the present embodiment, that it does not actively wake up birds of the flock of birds 11, but still provides sufficient intensity for awaken birds of the flock of birds 11 to find food and water in the space 9. Said value may alternatively be any other suitable lux value.

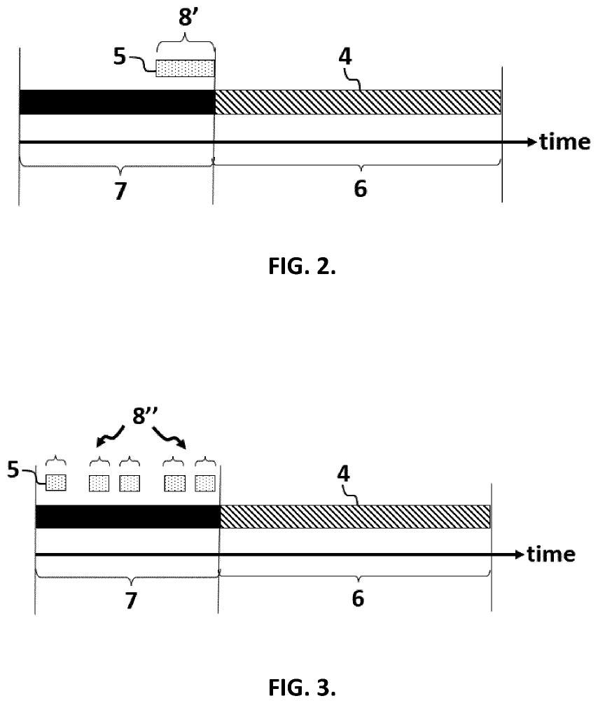

FIG. 2 refers to the same illumination system 10 as depicted in FIG. 1, but depicts schematically, by non-limiting example, a different operation according to the invention. Namely: The controller 3 is similarly configured to control the blue light source 2 to emit the monochromatic blue light 5 at a blue light intensity for at least one subperiod 8′ within the predetermined period of dark 7.

However, in the present embodiment, a subperiod 8′ of the at least one subperiod ends at a same time as the predetermined period of dark 7 and lasts for a duration equal to at most a quarter of the predetermined period of dark 7. Because said subperiod 8′ precedes and abuts the predetermined period of light 6 in time, the monochromatic blue light 5 is illuminating the space 9 just before the predetermined period of light 6 commences and the flock of birds 11 awaken (based on their circadian rhythm).

This is advantageous, because the predetermined period of light 6, in which feed and water are visible to the flock of birds 11, is extended in time to the predetermined period of dark 7. Birds of the flock of birds 11, which are awake before the predetermined period of light 6 commences, will have the opportunity to already start feeding and drinking, without the entrained circadian rhythm (i.e. day/night cycle) and their melatonin cycle are being disturbed. This reduces the amount of hungry and voraciously eating birds at the time the predetermined period of light 6 commences, thereby reducing crowding and injuries.

FIG. 3 refers to the same illumination system 10 as depicted in FIG. 1, but depicts schematically, by non-limiting example, a different operation according to the invention. Namely: The controller 3 is similarly configured to control the blue light source 2 to emit the monochromatic blue light 5 at a blue light intensity for at least one subperiod 8″ within the predetermined period of dark 7.

However, in the present embodiment, said at least one subperiod 8″ is a plurality of intermittent subperiods 8″ within the predetermined period of dark 7. Each of said plurality of intermittent subperiods 8″ may for example be predefined according to a schedule or may for example be triggered by a sensor. Said sensor may e.g. detect awaken birds. Said intermittent subperiods 8″ may also be scheduled such to induce a physiological response of the flock of chicken.

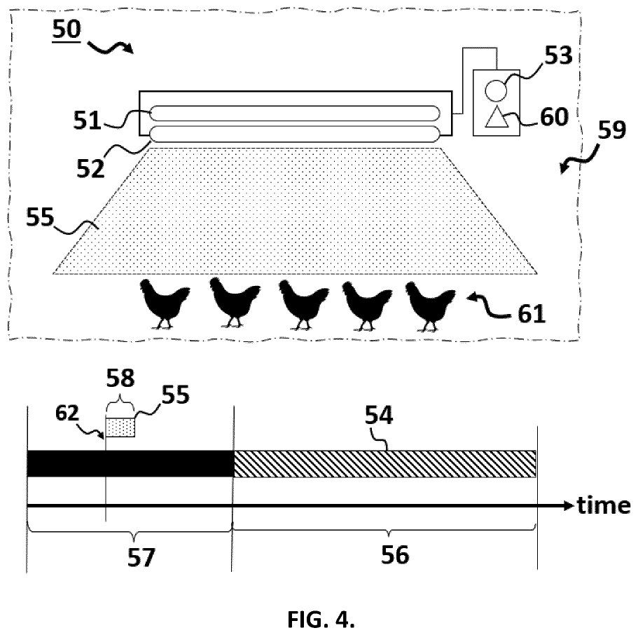

FIG. 4 depicts schematically, by non-limiting example, an illumination system 50 according to the invention.

The illumination system 50 comprises a visible light source 51, a blue light source 52, a controller 53, and a sensor unit 60. The controller 53 controls, in operation, the visible light source 51 and the blue light source 52. Here, the blue light source 52 and the visible light source 51 are depicted to be part of a same lighting device, such as a luminaire. Alternatively, said visible light source and said blue light source may be separate lighting devices.

The controller 53 is arranged remotely from said blue light source 52 and said visible light source 51, but in communication therewith via wired communication. For example via Power Line Communication (PLC). This may alternatively be via wireless communication, such as BLE, ZigBee, RF, Wi-Fi, VLC, Lo-Ra, etc.

The visible light source 51 is configured to emit visible light 56. More specifically, the controller 53 controls the visible light source 51 according to a predetermined circadian schedule, so as to entrain a circadian rhythm of the flock of birds 61. The predetermined circadian schedule consists of a predetermined period of light and a predetermined period of dark. These two periods recur every day within said circadian schedule. The controller 53 controls the visible light source 51 to emit the visible light 54 for a predetermined period of light 56, and not to emit the visible light 56 for a predetermined period of dark 57.

Here, merely as an example, the predetermined period of dark comprises a duration of 6 hours, while the predetermined period of light comprises a duration of 18 hours. Other alternative schedules of light and dark may be envisioned similarly. For example, the predetermined period of dark may be 12 hours, while the predetermined period of light may also be 12 hours.

Still referring to FIG. 4, the illumination system 50 is arranged in an indoor space 59 for rearing a flock of birds 61. Here, the flock of birds 61 is a flock of turkey, but may alternatively be any other bird suitable for rearing in an agricultural facility, such as chicken. The illumination system illuminates, in operation, the space 59 and thereby said flock of birds 61.

Hence, the visible light source 51 is able to render artificial lighting in the agriculture space 59 for entraining a circadian rhythm of said flock of birds 61, wherein said circadian rhythm is generally characterized by a 24-hour cycle comprising alternating periods of light (or: day) and periods of dark (or: night).

However, during such a period of dark, the flock of birds may not eat and drink, due to for example sleep or their inability to find food in the dark. This results in that their gastrointestinal tracts are nearly empty when the period of light (or: day) commences. Even after commencing the period of light within the circadian cycle, because the flock of birds may be very hungry, the flock of birds may exhibit a voraciously eating behaviour and may cause harmful crowding (at feeding throughs and water nipples).

Therefore, still referring to FIG. 4, the illumination system 50 according to the invention comprises the blue light source 52. The blue light source 52 is configured to emit monochromatic blue light 55. Here, the monochromatic blue light 55 comprises a dominant peak within the wavelength range of 405-480 nm.

Moreover, the illumination system 50 according to the invention comprises the sensor unit 60. The sensor unit 60 detects, in operation, during the predetermined period of dark 57, a property indicative of at least one bird of the flock of birds 61 being awake. Here, said sensor unit 60 is a motion detector for detecting the property of motion. Said property of motion is indicative of at least one bird of the flock of birds being awake. Here, the sensor unit 60 and the controller 53 are housed within a same device and in communication with each other, but the sensor unit and the controller may alternatively be separate devices.

Alternatively, said sensor unit 60 may comprise at least one of: a camera, a thermal camera, a microphone, a sensor arrangement for radiofrequency-based sensing, a PIR sensor, a thermopile array, a Single Pixel Thermopile, a range sensor, a VOC sensor, a pressure sensor.

More specifically, the controller 53 determines a number of birds being awake based on said property, namely said motion. Here, as an example, the sensor unit 60 detects said motion at a particular moment in time 62 during the predetermined period of dark 57. The motion is thereby indicative of twenty birds of the flock of birds 61 being awake. Hence, the controller 53 determines that twenty birds of the flock of birds 61 are awake.

Furthermore, the controller 53 determines a condition in which said number of birds being awake exceeds a predefined threshold number. Here, as an example, said predefined threshold number is ten. Therefore, said condition is determined, because the detected twenty birds exceed the predefined threshold number of ten. Alternatively, said predefined threshold number may be envisioned to be a different value, e.g. depending on the operations of the livestock facility or the type of the flock of birds.

Even further, the controller 53 controls the blue light source 52 to emit the monochromatic blue light 55 at a blue light intensity for at least one subperiod 58 within the predetermined period of dark 57 when said number of birds being awake exceeds the predefined threshold number, (or when said condition is determined). Since this is the case, the blue light source 52 is controlled to emit said monochromatic blue light 55.

Here, the at least one subperiod 58 is a subperiod 58. Thus, this subperiod 58 starts essentially at said particular moment in time 62 and lasts for a duration equal to one-sixth of the predetermined period of dark 57. Namely, in the present example, said subperiod 58 comprises a duration of one hour, as the predetermined period of time lasts for six hours. Alternatively, said duration may be equal to at most half of the predetermined period of dark, or at most a quarter of the predetermined period of dark, such as a tenth of the predetermined period of dark. Alternatively, in embodiments, said sensor unit may only be active during said predetermined period of dark.

In alternative examples, said sensor unit may detect said condition a plurality of times within the predetermined period of dark. Hence, each time said condition (of the number of birds being awake exceeding a predefined threshold number) is determined, the blue light source may be controlled to emit the monochromatic blue light for a respective subperiod.

Consequently, during the predetermined period of dark 57 the flock of birds 61 are provided with said monochromatic blue light 55, for a subperiod equal to one-sixth of the predetermined period of dark 57 after the sensor unit 60 has detected said property and the controller 53 has determined that the number of birds being awake exceeds the predefined threshold number.

Therefore, the illumination system 50 effectuates an increased health and wellbeing of the flock of birds 61, as partly mentioned above throughout the application.

Namely, since the blue light source 52 is controlled to emit the monochromatic blue light 55, a threshold number of awaken birds of the flock of birds 61 are still enabled to see within the space 59 and are thereby able to more efficiently feed and drink during the subperiod 58 within the predetermined period of dark 57, without the entrained circadian rhythm (i.e. day/night cycle) and their melatonin cycle are being disturbed.

Consequently, the birds still maintain their normal circadian rhythm, and thereby grow as scheduled, but with less stress of starvation during the predetermined period of dark 57, and with less harmful behaviour when the predetermined period of light 56 commences after the predetermined period of dark 57. Even further, since the blue light source 52 is controlled based on the detection of the sensor unit, the blue light source 52 may be more effectively and efficiently (i.e. e.g. in terms of power) be controlled.

Moreover, still referring to FIG. 4, as an example, the blue light intensity is 2 lux. Said blue light intensity is selected such, in the present embodiment, that it does not actively wake up birds of the flock of birds 61, but still provides sufficient intensity for the detected awaken birds of the flock of birds 61 to find food and water in the space 59. Said value may alternatively be any other suitable lux value, such as for example between 0.1 lux and 0.5 lux.

In an embodiment, not depicted, the illumination system of FIG. 1 and/or FIG. 4 comprises a first feed dispensing means arranged at a first region of the indoor space. The controller is then configured to control the blue light source to emit the monochromatic blue light at the blue light intensity for said subperiod within the predetermined period of dark to illuminate this first region of the indoor space. In the case of said illumination system of FIG. 4, this illumination is when said number of birds being awake exceeds the predefined threshold number.

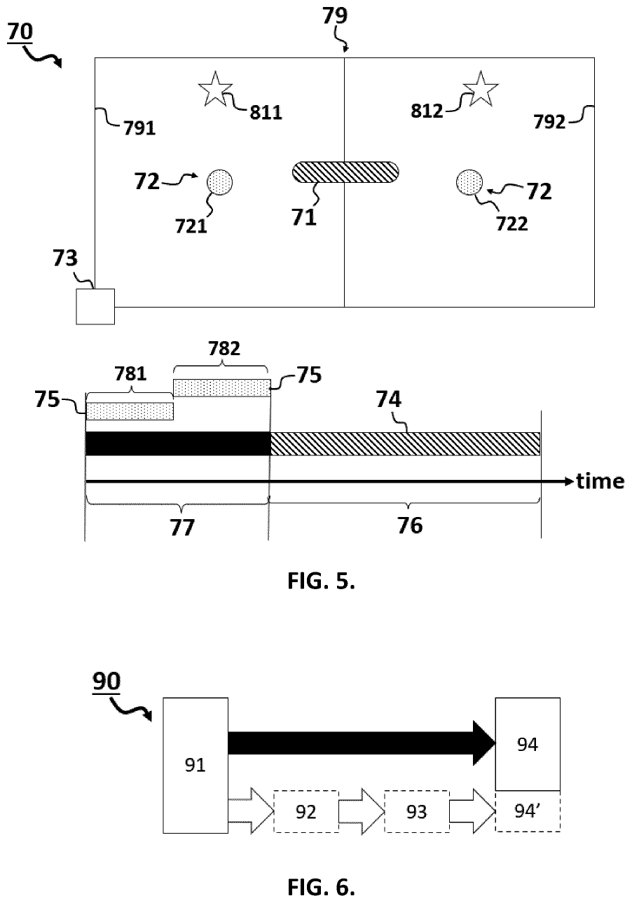

FIG. 5 depicts schematically, by non-limiting example, an embodiment of an illumination system 70 according to the invention. The illumination system 70 is arranged in an indoor space 79. The indoor space 80 is an agricultural facility for rearing a flock of birds (not depicted). Here, the flock of birds is a flock of chicken. The illumination system 70 illuminates, in operation, the indoor space 79 and thereby said flock of birds.

The indoor space 79 comprises a first region 791 and a second region 792. The first region 791 and the second region 792 are different. The first region 791 comprises, optionally, a first feed dispensing means 811. The second region 792 comprises, optionally, a second feed dispensing means 812. Hence, the illumination system 70 may in such embodiments also optionally comprise said first feed dispensing means 811 and said second feed dispensing means 812. Alternatively, said first region and said second region may at least partially overlap. Said space may alternatively, and optionally comprise at least one further region, such as a third region and a fourth region.

Still referring to FIG. 5, the illumination system 70 comprises a visible light source 71, a blue light source 72, and a controller 73. The controller 73 controls, in operation, the visible light source 71 and the blue light source 72. The controller 73 is thereby arranged remotely from said blue light source 72 and said blue light source 71, but in wireless communication therewith via a wireless communication modality, such as BLE, ZigBee, RF, Wi-Fi, VLC, Lo-Ra, etc. Alternatively, said controller and visible light source and blue light source(s) may be via a wired connection.

The visible light source 71 is configured to emit visible light 74 into the space 79. That is: both the first region 791 of the space 79 and the second region 792 of the space 79. More specifically, the controller 73 controls the visible light source 71 according to the predetermined circadian schedule, so as to entrain a circadian rhythm of the flock of birds. This is done by the controller 73 controlling the visible light source 71 to emit the visible light 74 for a predetermined period of light 76, and not to emit the visible light 74 for a predetermined period of dark 77.

Here, merely as an example, the predetermined period of dark 77 comprises a duration of 8 hours, while the predetermined period of light 76 comprises a duration of 16 hours, (in total). Other alternative schedules of light and dark may be envisioned similarly.

Hence, the visible light source 71 is able to render artificial lighting in the space 79 for entraining a circadian rhythm of said flock of birds, wherein said circadian rhythm is generally characterized by a 24-hour cycle comprising alternating periods of light (or: day) and periods of dark (or: night).

Still referring to FIG. 5, the illumination system 70 according to the invention comprises the blue light source 72. The blue light source 72 is configured, in operation, to emit monochromatic blue light 75 into said space 79. Here, the monochromatic blue light 75 comprises a peak wavelength within the wavelength range 405-480 nm.

Moreover, in the present embodiment, the blue light source 72 is an array of light source units. Such a light source unit may e.g. be a luminaire. Namely, the blue light source 72 comprises a first blue light source unit 721, and a second blue light source unit 722, both configured to emit said monochromatic blue light 75. The first blue light source unit 721 is arranged to illuminate the first region 791 of the space 79; and the second light source unit 722 is arranged to illuminate the second region 792 of the indoor space 79.

In alternatively examples, the blue light source may be a single luminaire that is configured to separately illuminate at least one of a plurality of regions within said space. Such a luminaire may therefore comprise at least one blue light source unit, at least one optic, and/or at least one beam steering means.

More specifically, still referring to the embodiment depicted in FIG. 5, the controller 73 controls the first blue light source unit 721 to emit the monochromatic blue light 75 at a first blue light intensity during a first subperiod 781 within the predetermined period of dark 77 to illuminate the first region 791 of the indoor space 79. The controller 73 similarly controls the second blue light source unit 722 to emit the monochromatic blue light 75 at a second blue light intensity during a second subperiod 782 within the predetermined period of dark 77 to illuminate a second region 792 of the space 79. Still referring to FIG. 6, the first subperiod 781 and the second subperiod 782 are non-overlapping in time. Here, the first subperiod precedes 781 the second subperiod 782. Here, the first subperiod 781 abuts the second subperiod 782. Alternatively, said first subperiod and said second subperiod may be independent from each other, and their properties (such as duration and begin and end points in time) envisioned such as the examples provided for subperiods in this application.

In the present example, said first subperiod 781 and said second subperiod 782 comprise both a duration equal to half the predetermined period of dark 77. Since the predetermined period of dark comprises a duration of 8 hours in the present example, said duration of the first subperiod 781 and the second subperiod 782 are both 4 hours. Consequently, during the predetermined period of dark 77 the flock of birds are also provided with said monochromatic blue light 75, but first in the first region 791 and subsequently in the second region 792. This advantageously enables a spatial control of the monochromatic blue light 75 within the space 79, for example to provide monochromatic blue light 75 to the said first feed dispensing means 811 and/or said second feed dispensing means 812.

For example, in alternative aspects, a first sensor unit observing the first region may trigger the first blue lighting unit to illuminate the first region with the first feed dispensing means; and a second sensor unit observing the second region may trigger the second blue lighting unit to illuminate the second region with the second feed dispensing means.

Hence, since the blue light source 72 is controlled to emit the blue light 75, birds of the flock of birds-when awaken and hungry-are still able to see within the respective regions 791, 792 of said space 79; and are thereby able to more efficiently feed and drink during the predetermined period of dark 7, without the entrained circadian rhythm (i.e. day/night cycle) and their melatonin cycle are being disturbed.

Consequently, the birds still maintain their normal circadian rhythm, and thereby grow as scheduled, but with less stress of starvation during the predetermined period of dark 77, and with less harmful behaviour when the predetermined period of light 76 commences after the predetermined period of dark 77.

Moreover, still referring to FIG. 5, the blue light intensity is 5 lux. Said blue light intensity is selected such, in the present embodiment, that it does not actively wake up birds of the flock of birds, but still provides sufficient intensity for awaken birds of the flock of birds to find food and water in the respective regions 791, 792 of said space 79. Said value may alternatively be any other suitable lux value.

Therefore, the illumination system according to the present invention may also actively awake birds within the predetermined period of dark, such that said awaken birds may be able to feed during the predetermined period of dark, so as to mitigate the disadvantages and problems mentioned above. Even further, by regionally targeting the blue light for awaking the chicken, the feeding of the flock within the predetermined period of dark may be controlled and regulated accordingly. For example, a first part of the flock of birds may be awaken, and provided with visible light to find feed, during a first subperiod; and a second part of the flock of birds may be awaken, and provided with visible light to find feed, during a second subperiod; wherein the first subperiod and second subperiod may be different. This will also regulate crowding during feeding within the predetermined period of dark.

FIG. 6 depicts schematically, by non-limiting example, a method 90 of illuminating a bird during at least one growth phase. The method may be performed with the illumination system according to the present invention.

The method 90 comprises a step 91 of emitting visible light for a predetermined period of light during a full-day period, and not to emit visible light for a predetermined period of dark during the remainder of said full-day period, so as to entrain a circadian rhythm of the bird. Hence, the visible light is emitted according to a predetermined circadian schedule for entraining a circadian rhythm of the bird.

The method 90 further comprises a step 94 of emitting monochromatic blue light at a blue light intensity for at least one subperiod within the predetermined period of dark.

Said bird may alternatively be a flock of birds. Said bird may be a bird in the incubation phase, still comprised within an egg. Hence, the method may be arranged for illuminating a bird within a space, said space being for example an egg or an agricultural space for rearing birds.

In further embodiments, the method may optionally comprise a step 92 of detecting, during the predetermined period of dark, a property indicative of at least one bird of the flock of birds being awake; and a step 93 of determining a number of birds being awake based on said property. Then, the method may comprise a step 94′ of controlling the blue light source to emit the monochromatic light at the blue light intensity for a subperiod within the predetermined period of dark when said number of birds being awake exceeds a predefined threshold number.

Alternatively, the method may comprise the steps of detecting, during the predetermined period of dark, a property indicative of at least one bird of the flock of birds being awake; and controlling the blue light source to emit the monochromatic blue light at the blue light intensity for a subperiod within the predetermined period of dark when said property is detected.

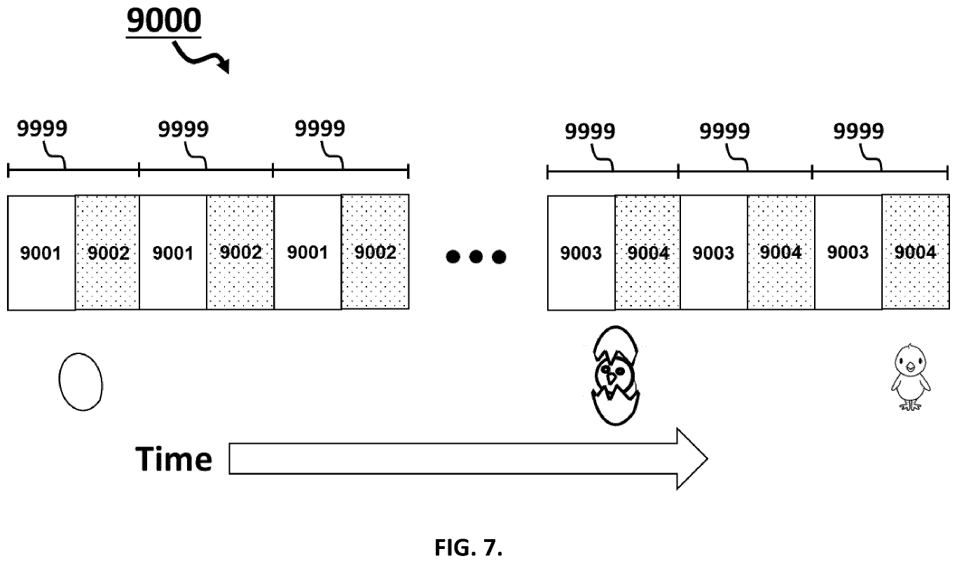

FIG. 7 depicts schematically, by non-limiting example, an embodiment of a method 9000 of illuminating a bird during at least one growth phase according to the invention. Each of said growth phase of the at least one growth stage comprises a range of days. Here, the growth phase is a pre-hatch phase and/or a post-hatch phase. The method 9000 is repeatedly performed for each day in said range of days.

More specifically, for each day in said range of days of the pre-hatch phase, the method comprises a step (9001) of emitting visible light for a predetermined period of light during a full-day period 9999, and not to emit visible light for a predetermined period of dark during the remainder of said full-day period 9999, so as to entrain a circadian rhythm of the bird; and a step (9002) of emitting monochromatic blue light at a blue light intensity for at least one subperiod within the predetermined period of dark.

Moreover, for each day in said range of days of the post-hatch phase, the method further comprises, a step (9003) of emitting visible light for a predetermined period of light during a full-day period 9999, and not to emit visible light for a predetermined period of dark during the remainder of said full-day period 9999, so as to entrain a circadian rhythm of the bird; and a step (9004) of emitting monochromatic blue light at a blue light intensity for at least one subperiod within the predetermined period of dark.

In the present embodiment, by way of example, the pre-hatch phase comprises a range of days of zero and twenty-one days from fertilization of the bird (i.e. the bird embryo) or from placing an egg containing the bird in an incubator. It is found, during the incubation phase, which is largely pre-hatch, that incubating with the predetermined circadian schedule, wherein monochromatic blue light is emitted at the blue light intensity for at least one subperiod within the predetermined period of dark improves hatchability of fertile eggs and renders an increased weight of hatched chicks

In the present embodiment, by way of example, the post-hatch phase comprises a range of days of fourteen and forty days from hatching of said bird. Other example ranges may be envisioned similarly.

It is found that post-hatch birds feed and drink more effectively within the dark/night period of their circadian rhythm with the presence of the monochromatic blue light, without disrupting their entrained circadian rhythm (i.e. day/night cycle). This renders an increased weight gain, improved Feed Conversion Ratio, and reduced mortality for post-hatch birds.

Still referring to the method 9000 as depicted in FIG. 7, the blue light intensity is at most 5 lux, namely 1 lux in the present embodiment. The at least one subperiod is one subperiod, wherein said one subperiod lasts the whole predetermined period of dark. Moreover, the predetermined period of light is twelve hours, and the predetermined period of dark is twelve hours, together forming the full-day period 9999. Optionally, the visible light may comprise a dominant peak at a wavelength range between 450-480 nm and a dominant peak at a wavelength range between 515-545 nm.

The present invention is particularly advantageous for rearing of broiler (meat) chickens, especially chicks. These birds are very fast growing and require a large quantity of feed to allow that growth. The present invention will allow the birds to eat and drink throughout the ‘night’ (i.e. the predetermined period of dark, i.e. the scotopic phase of the circadian schedule) without adversely affecting their melatonin cycle or circadian rhythm, thereby lowering stress and increasing production.

The present invention may also be advantageous for rearing laying hens and breeder hens. Namely, laying hens and breeder hens prefer to roost on a perch at night (i.e. the predetermined period of dark), providing blue light will allow them to see the perch even during the night-time period. This may reduce the incidence of ‘falls’, where the bird attempts to land on the perch but misses, often leading to bruising or keel bone fractures. Hence, the present invention even further promotes animal health and wellbeing.

As mentioned, the present invention may also be advantageous for bird embryos pre-hatch, as the dim, monochromatic blue light results in heavier chicks at hatch and improved hatchability.

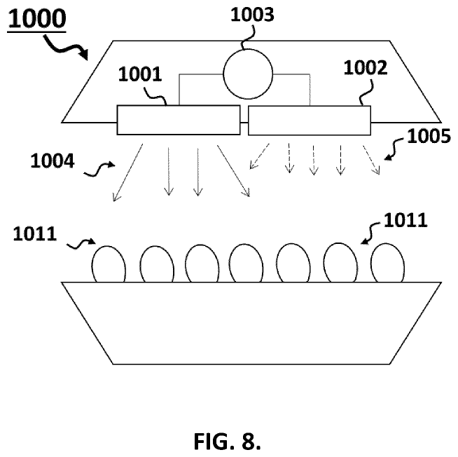

FIG. 8 depicts schematically, by non-limiting example, an embodiment of an incubator 1000 according to the invention. The incubator 1000 comprises the illumination system according to the invention. The incubator 1000 is arranged to incubate fertilized bird eggs 1011. The fertilized bird eggs, or bird eggs, comprise a respective bird.

Still referring to FIG. 8, the incubator 1000 comprises a visible light source 1001 configured to emit visible light 1004. The incubator 1000 comprises a blue light source 1002 configured to emit monochromatic blue light 1005. The incubator 1000 comprises a controller 1003 configured to control the visible light source 1001 according to a predetermined circadian schedule for entraining a circadian rhythm of a bird. Here, the bird (embryo) is in the eggs 1011 in the incubator 1000. Said predetermined circadian schedule consists of a predetermined period of light and a predetermined period of dark. The controller 1000 is configured to: (i) control the visible light source 1001 to emit the visible light 1004 for the predetermined period of light. The visible light source 1004 is thereby emitted at a visible light intensity. The controller 1000 is further configured not to emit the visible light 1004 for the predetermined period of dark. The controller 1000 is moreover configured to control the blue light source 1002 to emit the monochromatic blue light 1005 at a blue light intensity for at least one subperiod within the predetermined period of dark. Here, the at least one subperiod is one subperiod, wherein said one subperiod lasts the whole predetermined period of dark.

Here, the blue light intensity is at most 5 lux, such as for example 0.5 lux. The visible light intensity is for example 100 lux. Hence, the visible light intensity is at least a factor 20 larger than the blue light intensity.

Referring to FIG. 8, the bird eggs 1011 may be incubated during a growth phase for a range of days. The growth phase may be incubation, or alternatively pre-hatch phase. Here, the range of days may for example be from day 1 to day 28 from fertilization of the bird (i.e. the bird embryo) in the eggs 1011 or from placing said eggs 1011 containing the birds in the incubator 1000. Each day thereof comprises said predetermined period of light during and predetermined period of dark, so as to entrain the circadian rhythm of the birds in the eggs 1011.

1. An illumination system comprising:
a visible light source configured to emit visible light;
a blue light source configured to emit monochromatic blue light;
a controller configured to control the visible light source according to a predetermined circadian schedule for entraining a circadian rhythm of a bird;
wherein the predetermined circadian schedule consists of a predetermined period of light and a predetermined period of dark;
wherein the controller is configured to:
(i) control the visible light source to emit the visible light for the predetermined period of light, and not to emit the visible light for the predetermined period of dark;
(ii) control the blue light source to emit the monochromatic blue light at a blue light intensity for at least one subperiod within the predetermined period of dark;
wherein the illumination system is arranged for illuminating a flock of birds;
wherein the illumination system comprises a sensor unit configured to detect, during the predetermined period of dark, a property indicative of at least one bird of the flock of birds being awake;
wherein the controller is configured to: control the blue light source to emit the monochromatic blue light at the blue light intensity for a subperiod within the predetermined period of dark when said property is detected; OR
wherein the controller is configured to:
determine a number of birds being awake based on said property, and
control the blue light source to emit the monochromatic blue light at the blue light intensity for a subperiod within the predetermined period of dark when said number of birds being awake exceeds a predefined threshold number.

2. The illumination system according to claim 1, wherein the monochromatic blue light intensity is at most 5 lux.

3. The illumination system according to claim 1, wherein said subperiod lasts for a duration equal to at most a half of the predetermined period of dark.

4. The illumination system according to claim 1, wherein the sensor unit comprises at least one of: a camera, a thermal camera, a microphone, a motion sensor, a sensor arrangement for radiofrequency-based sensing, a PIR sensor, a thermopile array, a Single Pixel Thermopile, a range sensor, a VOC sensor, a pressure sensor.

5. The illumination system according to claim 1, wherein the controller is configured to obtain an input signal indicative of the predetermined circadian schedule, and to determine the predetermined circadian schedule based on said input signal.

6. The illumination system according to claim 5, wherein the input signal is a user input signal.

7. The illumination system according to claim 1, wherein the illumination system comprises a first feed dispensing means arranged at a first region of the indoor space;
wherein the controller is configured to control the blue light source to emit the monochromatic blue light at the blue light intensity for the subperiod within the predetermined period of dark to illuminate the first region of the indoor space.

8. The illumination system according to claim 7, wherein the illumination system comprises a first feed dispensing means arranged at the first region of the indoor space.

9. A method of illuminating a bird during at least one growth phase, wherein the method comprises:
emitting visible light for a predetermined period of light during a full-day period, and not to emit visible light for a predetermined period of dark during the remainder of said full-day period, so as to entrain a circadian rhythm of the bird;
emitting monochromatic blue light at a blue light intensity for at least one subperiod within the predetermined period of dark;
detecting, during the predetermined period of dark, a property indicative of at least one bird of the flock of birds being awake;
controlling the blue light source to emit the monochromatic blue light at the blue light intensity for a subperiod within the predetermined period of dark when said property is detected; OR
determining a number of birds being awake based on said property, and
controlling the blue light source to emit the monochromatic blue light at the blue light intensity for a subperiod within the predetermined period of dark when said number of birds being awake exceeds a predefined threshold number.

10. The method according to claim 9, wherein the predetermined period of light is twelve hours, and wherein the predetermined period of dark is twelve hours.

11. The method according to claim 9, wherein the visible light comprises a dominant peak at a wavelength range between 450-480 nm and a dominant peak at a wavelength range between 515-545 nm.

12. The method according to claim 9, wherein the monochromatic blue light intensity is at most 5 lux.

13. The method according to claim 9, wherein the at least one subperiod is one subperiod, wherein said one subperiod lasts the whole predetermined period of dark.

14. The method according to claim 9, wherein the at least one growth phase is at least one of: a pre-hatch phase, a hatching phase, a post-hatch phase.

15. (canceled)
